(19) United States
(12) Patent Application Publication
(10) Pub. No.: US 2022/0143373 A1
(43) Pub. Date: May 12, 2022

(54) SYSTEMS, CATHETERS, AND METHODS FOR TREATING ALONG THE CENTRAL NERVOUS SYSTEM

(71) Applicant: MINNETRONIX NEURO, INC., ST. PAUL, MN (US)

(72) Inventors: BRYN ANDREW MARTIN, MOSCOW, ID (US); MOHAMMADREZA KHANI, MOSCOW, ID (US); AARON R. MCCABE, EDINA, MN (US); LAURA MARIE ZITELLA VERBICK, NORTH ST. PAUL, MN (US)

(73) Assignee: MINNETRONIX NEURO, INC., ST. PAUL, MN (US)

(21) Appl. No.: 17/583,814

(22) Filed: Jan. 25, 2022

Related U.S. Application Data
(63) Continuation of application No. PCT/US2020/043551, filed on Jul. 24, 2020.
(60) Provisional application No. 62/878,587, filed on Jul. 25, 2019.

(51) Int. Cl. A61M 27/00

(52) U.S. Cl. CPC A61M 27/006; A61M 2025/0004; A61M 2027/004

(57) ABSTRACT

Systems, catheters, and methods for accessing and treating along the central nervous system are disclosed. A system for processing cerebrospinal fluid is disclosed. The system may include a catheter having a proximal subassembly and a distal subassembly. A pump and filtration system may be coupled to the catheter. An infusion lumen for infusing cerebrospinal fluid filtered by the pump and filtration system may be defined along the distal subassembly. The distal subassembly may define a plurality of infusion openings in fluid communication with the infusion lumen. The infusion openings may increase in size distally along the distal subassembly.

### CROSS-REFERENCE TO RELATED APPLICATIONS

This application is a continuation of International Application No. PCT/US2020/43551, filed Jul. 24, 2020, which claims the benefit of priority under 35 U.S.C. § 119 to U.S. Provisional Application Ser. No. 62/878,587 filed Jul. 25, 2019, the entirety of which is incorporated herein by reference.

### TECHNICAL FIELD

The present disclosure relates to systems, catheters, and methods for treating along the central nervous system.

### BACKGROUND

A wide variety of medical devices have been developed for medical use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. A system for processing cerebrospinal fluid is disclosed. The system comprises: a catheter having a proximal subassembly and a distal subassembly; a pump and filtration system coupled to the catheter; wherein an infusion lumen for infusing cerebrospinal fluid filtered by the pump and filtration system is defined along the distal subassembly; wherein the distal subassembly defines a plurality of infusion openings in fluid communication with the infusion lumen; and wherein the infusion openings increase in size distally along the distal subassembly.

Alternatively or additionally to any of the embodiments above, at least some of the infusion openings have a round shape.

Alternatively or additionally to any of the embodiments above, at least some of the infusion openings have a non-circular shape.

Alternatively or additionally to any of the embodiments above, all of the infusion openings have the same shape.

Alternatively or additionally to any of the embodiments above, at least some of the infusion openings differ in shape.

Alternatively or additionally to any of the embodiments above, the plurality of infusion openings include a row of axially-aligned infusion openings.

Alternatively or additionally to any of the embodiments above, at least some of the plurality of infusion openings extend circumferentially about the distal subassembly.

Alternatively or additionally to any of the embodiments above, the proximal subassembly includes a plurality of aspiration openings.

Alternatively or additionally to any of the embodiments above, at least some of the aspiration openings have a round shape.

Alternatively or additionally to any of the embodiments above, at least some of the aspiration openings have a non-circular shape.

Alternatively or additionally to any of the embodiments above, all of the aspiration openings have the same shape.

Alternatively or additionally to any of the embodiments above, at least some of the aspiration openings differ in shape.

Alternatively or additionally to any of the embodiments above, the plurality of aspiration openings include a row of axially-aligned aspiration openings.

Alternatively or additionally to any of the embodiments above, at least some of the plurality of aspiration openings extend circumferentially about the proximal subassembly.

Alternatively or additionally to any of the embodiments above, the aspiration openings increase in size distally along the proximal subassembly.

Alternatively or additionally to any of the embodiments above, the aspiration openings increase in size proximally along the proximal subassembly.

A system for processing cerebrospinal fluid is disclosed. The system comprises: a catheter having a proximal subassembly and a distal subassembly; a pump and filtration system coupled to the catheter; wherein an aspiration lumen for aspirating cerebrospinal fluid is defined along the distal subassembly; wherein the proximal subassembly defines a plurality of aspiration openings in fluid communication with the aspiration lumen; and wherein the aspiration openings change in size along the proximal subassembly.

Alternatively or additionally to any of the embodiments above, the aspiration openings increase in size distally along the proximal subassembly.

Alternatively or additionally to any of the embodiments above, the aspiration openings increase in size proximally along the proximal subassembly.

Alternatively or additionally to any of the embodiments above, at least some of the aspiration openings have a round shape.

Alternatively or additionally to any of the embodiments above, at least some of the aspiration openings have a non-circular shape.

Alternatively or additionally to any of the embodiments above, all of the aspiration openings have the same shape.

Alternatively or additionally to any of the embodiments above, at least some of the aspiration openings differ in shape.

Alternatively or additionally to any of the embodiments above, the plurality of aspiration openings include a row of axially-aligned aspiration openings.

Alternatively or additionally to any of the embodiments above, at least some of the plurality of aspiration openings extend circumferentially about the proximal subassembly.

Alternatively or additionally to any of the embodiments above, the distal subassembly includes a plurality of infusion openings.

A system for processing cerebrospinal fluid is disclosed. The system comprises: a catheter having a proximal subassembly and a distal subassembly; a pump and filtration system coupled to the catheter; wherein an infusion lumen for infusing cerebrospinal fluid filtered by the pump and filtration system is defined along the distal subassembly; wherein the distal subassembly defines a plurality of infusion openings in fluid communication with the infusion lumen; wherein the infusion openings increase in size distally along the distal subassembly; wherein an aspiration lumen for aspirating cerebrospinal fluid is defined along the distal subassembly; and wherein the proximal subassembly defines a plurality of aspiration openings in fluid communication with the aspiration lumen.

Alternatively or additionally to any of the embodiments above, the aspiration openings increase in size distally along the proximal subassembly.

Alternatively or additionally to any of the embodiments above, the aspiration openings increase in size proximally along the proximal subassembly.

A system for processing cerebrospinal fluid is disclosed. The system comprises: a catheter having a proximal subassembly and a distal subassembly; a pump and filtration system coupled to the catheter; wherein an infusion lumen for infusing cerebrospinal fluid filtered by the pump and filtration system is defined along the distal subassembly; wherein the distal subassembly defines a plurality of infusion openings in fluid communication with the infusion lumen; wherein the infusion openings increase in size distally along the distal subassembly; wherein an aspiration lumen for aspirating cerebrospinal fluid is defined along the distal subassembly; wherein the proximal subassembly defines a plurality of aspiration openings in fluid communication with the aspiration lumen; wherein the aspiration openings change in size along the proximal subassembly.

Alternatively or additionally to any of the embodiments above, the aspiration openings increase in size distally along the proximal subassembly.

Alternatively or additionally to any of the embodiments above, the aspiration openings increase in size proximally along the proximal subassembly.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates a Y-connector portion, a proximal subassembly, and a distal subassembly of a catheter according to certain implementations.

FIG. 2 illustrates a sectional view taken from the region of the catheter of FIG. 1 marked with cutting plane line A-A.

FIG. 3 illustrates a sectional view taken from the region of the catheter of FIG. 1 marked with cutting plane line B-B.

FIG. 4 illustrates an enlarged, detail view of a portion of the Y-connector of the catheter of FIG. 1.

FIG. 5 illustrates the location of position markers on a catheter according to certain implementations.

FIG. 6 illustrates a sectional view taken from the region of the catheter of FIG. 5 marked with the cutting plane line J-J.

FIG. 7 illustrates a portion of a catheter near the joining of a proximal subassembly and a distal subassembly according to certain implementations.

FIG. 8 illustrates a portion of a proximal subassembly according to certain implementations.

FIG. 9 illustrates a detail view of the proximal subassembly of FIG. 8.

FIG. 10 illustrates a sectional view taken from the region of the proximal subassembly of FIG. 8 marked with the cutting plane line A-A.

FIG. 11 illustrates a detail view of a portion of the proximal subassembly of FIG. 9 taken from the view of line D-D.

FIG. 12 illustrates a sectional view taken from the region of the proximal subassembly of FIG. 8 marked with the cutting plane E-E.

FIG. 13 illustrates a portion of a distal subassembly according to certain implementations.

FIG. 14 illustrates a detailed portion of the distal subassembly of FIG. 13.

FIG. 15 illustrates a detailed portion of the distal subassembly of FIG. 13.

FIG. 16 illustrates a sectional view taken from the region of the distal subassembly of FIG. 13 marked with the cutting plane A-A.

FIG. 17 schematically illustrates an example pump system.

FIG. 18 illustrates a portion of an example catheter.

FIG. 19 illustrates a portion of an example catheter.

FIG. 20 illustrates a portion of an example catheter.

FIG. 21 illustrates a portion of an example catheter.

FIG. 22 illustrates a portion of an example catheter.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAIL DESCRIPTION

Cerebrospinal fluid (CSF) is a generally clear, colorless fluid with viscosity similar to water that is produced within the choroid plexus located in the ventricles of the brain. Total CSF volume has been estimated to range from approximately 150 to 300 milliliters in healthy adults. The choroid plexus is believed to produce approximately 500 milliliters of CSF daily in order to accommodate flushing or recycling of CSF to remove toxins and metabolites. The total volume of CSF is replenished several times per day or possibly more during sleep cycles and other activities. CSF also serves to float the delicate brain tissue by the Archimedes principle, and it protects the brain from sudden movements by cushioning the tissue. From the choroid plexus, CSF flows slowly through a series of openings into the space surrounding the brain and spinal column, and then into the body through multiple outflow pathways that include arachnoid granulations, cribuform plate, dural lymphatics, spinal cord nerve root sleeves, and possibly other pathways within the brain tissue. CSF is found in the space between the pia mater and the arachnoid mater, known as the subarachnoid space and also located within the ventricular system of the brain and in a series of cisterns located external to the brain. In addition to the net production and absorption of CSF flow, the CSF oscillates with a back-and-forth motion in synchrony with the cardiac and respiratory cycle. The magnitude of these oscillations is variable depending on the specific region of CSF. CSF flow can also be intermittently altered based on various maneuvers such as valsalva, coughing, sneezing, playing a musical instrument, and athletic activities. CSF pressure in a healthy adult is approximately 10 millimeters of mercury in the supine position. CSF pressure is altered in the standing position by hydrostatic pressure gradient along CSF system and can also be transiently affected by maneuvers such as coughing.

Research has indicated that alterations of the biochemical composition of CSF can be indicative and/or involved in the pathological processes of a plethora of central nervous system disease states. For example, in the event of a stroke or other brain trauma, blood can enter the CSF system leading to subsequent injury to the brain due to blood clotting and other biological processes. In context of amyotrophic lateral sclerosis, several chemicals (inflammatory proteins or cytokines such as CHIT1) have been found to be abnormally elevated potentially contributing to the disease pathology. Similarly, in multiple sclerosis proteins, cytokines and chemokines have been found to be elevated and potentially underlying disease progression. As such, in principle, it could be beneficial to remove CSF with abnormal biochemical composition; however, direct removal of CSF is limited as only relatively small amounts can be safely removed. Thus, it can be desirable to remove the CSF from one location (e.g., the cervical region of the spine, or a brain ventricle), alter it (e.g., filter), and return it to the CSF space at a second location (e.g., the lumbar region of the spine). This process can be used to remove the unwanted biochemical products while maintaining similar total CSF volume. However, accurate delivery of medical instruments to the CSF space can be challenging.

The present disclosure relates to removal, exchange and recirculation of cerebrospinal fluid (CSF). Devices, systems and methods disclosed herein are used to safely and efficiently navigate the space at and around the brain and spinal cord where the CSF flows through the body, also known as the CSF space. Specialized devices and systems are useful and sometimes necessary to navigate the CSF space due to the difficult points of entry and exit and the potentially life-threatening consequences if a mistake is made.

Neurapheresis may be understood to be the modification of materials (e.g. removal of microrganisms, cells, viruses, foreign material, drugs, combinations thereof, and the like, or circulation and/or addition of materials such as pharmacologic agents) from CSF. This and other therapeutic techniques can be used to treat a number of neurological diseases or conditions, such as Alzheimer' s Disease, Parkinson's Disease, Huntington's Disease, Amyotrophic Lateral Sclerosis (ALS), Encephalitis from various causes, Meningitis from various causes, Guillain-Barré Syndrome (GBS), Multiple Sclerosis (MS), HIV-associated neurocognitive disorders, Spinal Cord Injury, Traumatic Brain Injury, cerebral vasospasm, stroke and other diseases or conditions. In addition, Neurapheresis can be used during open or endoscopic spine surgery or brain surgery, for example to remove blood that may get in the CSF during the surgery.

Focusing on filtration: the purification, conditioning, and/or compound removal schema can be tailored to a specific disease or group of diseases as suitable, including based on a number of features, such as size, affinity, biochemical properties, temperature, and other features. Purification schema may be based on diffusion, size-exclusion, ex-vivo immunotherapy using immobilized antibodies or antibody fragments, hydrophobic/hydrophilic, anionic/cationic, high/low binding affinity, chelators, anti-bacterial, anti-viral, anti-DNA/RNA/amino acid, enzymatic, and magnetic and/or nanoparticle-based systems. The system can be adjustable to a broad range of biologic parameters and flows.

With regard to a Neurapheresis system in particular, the disclosed system can be used to safely and quickly access the CSF space with minimal disturbance to the CSF flow. The systems and devices disclosed herein provide a safe and rapid flow circuit and provide filtration.

A Neurapheresis system should provide for the exchange, removal, and/or recirculation of CSF, safely and efficiently. The systems and devices disclosed herein may be used in a Neurapheresis system.

The systems and devices disclosed herein can be used to access the CSF space to remove the CSF from one location (e.g., the cervical or lumbar region of the spine, or a brain ventricle), filter or otherwise treat it, and return it to the CSF space, including at a second location (e.g., the cervical or lumbar region of the spine or a brain ventricle), safely and efficiently. In various aspects, the systems and devices disclosed herein maintain the endogenous intracranial or intraspinal pressure within a physiological range, for example, from about 5 to about 20 mm Hg or from about 0 to about 10 mm Hg or from about −5 to about 10 mm Hg or from about −5 to about 25 mm Hg. In some of these and in other instances, the present system may be used to help resolve a spinal headache, for example due to hydrocephalus (abnormal accumulation of CSF in the ventricles of the brain), by restoring pressure that was abnormal. For example, the present system may also be used to reduce spinal headaches caused by low pressure (e.g., due to overdrainage, herniation, etc.). In some aspects, the system may include sensors within the catheter or within the flow circuit to detect clogs or blockages in the system, thereby providing closed loop pressure control. In various aspects, the systems and devices disclosed herein also help the system to perform efficiently by reducing or eliminating recirculating flow loops. The systems and devices maintain spacing between the inlet and outlet, for example, between about 10 cm to about 40 cm. In certain implementations, the spacing is between about 10 cm and about 30 cm. The inlets and outlets are located in places in the CSF space so that turning on the pump or otherwise creating positive or negative pressure in the system will not cause or encourage tissue being drawn into the catheter. In some aspects, the inlets and outlets are placed near the lumbar/cervical cisterns to prevent tissue from being drawn into the catheter. In some aspects, there may also be multiple holes along the inlet and outlet for redundancy in case there is tissue blocking some number of holes. In certain implementations, a particular coil pitch of a coiled wire within the catheter may be selected in order to reduce kinking of the catheter. In certain aspects, the inlet-outlet spacing may be selected to be maximized while staying below the level of a cervical region of a patient. In certain aspects, the inlet-outlet spacing may be selected based on vertebral spacing. For example, the spacing may be selected so that the inlet-outlet spacing is between the lengths of approximately 5 vertebrae and approximately 12 vertebrae. In certain implementations, a spacing of approximately 10 vertebrae may be selected; however, other configurations (such as those described elsewhere in the specification) may be utilized. When designing such spacing, it may be assumed that a vertebra is approximately 2-3 cm in length, however, other measurements and designs may be used. In certain implementations, a particular size, shape, and/or other configuration of a lumen may be selected to facilitate catheter unblocking and/or the ability of the catheter to resist blockage. For example, a proximal outer diameter of a lumen of between approximately 0.060 inches and approximately 0.070 inches and a proximal inner diameter of between approximately 0.025 inches and 0.060 inches may be selected; however, other configurations (such as those described elsewhere in the specification) may be utilized.

The disclosed systems and devices are used to access the CSF space and may be used at any access point in the cervical (C1-C7), thoracic (T1-T12), or lumbar region (L1-L5) of the vertebral column. An access site in the cervical region may be used to access the ventricular system in the brain. In one embodiment, the system and device are used to access the lumbar region. In some embodiments, the inlets and outlets are located in places in the spine such that the drainage process will not cause tissue to be drawn into the catheter. For example, when a patient is lying on a table, entry may be made at a suitable angle, such as, for example, about 90 degrees, to access the spine. A traditional catheter must be pushed through a 90-degree bend at the L4-L6 region. The catheters and related delivery devices disclosed herein may be curved such that they can access and navigate this angled bend more easily and efficiently.

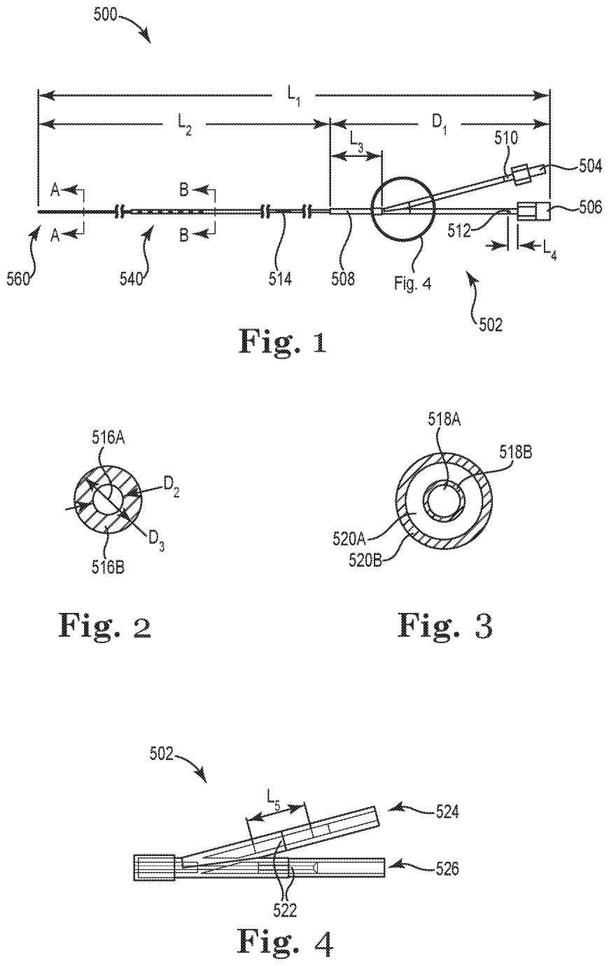

FIGS. 1-16 illustrate overall views, proximal subassembly views, and distal subassembly views of an embodiment of a catheter 500 according to certain implementations. FIG. 1 illustrates a Y-connector portion 502, a proximal subassembly 540, and a distal subassembly 560. The Y-connector portion 502 may include connectors 504, 506, features 508, 510, 512, position marker 514, and other components. The connectors 504, 506 may take various forms. For example, as illustrated, the connectors 504, 506 are female and male Luer-lock connectors, respectively. The features 508, 510, 512 may be strain relief and kink resistance features, for example, as described above with reference to strain relief and kink resistance feature 60. The feature 508 may be configured to allow flex or deformation of the catheter 500 at portions near a central meeting point of the Y-connector 502. The features 510, 512 may be configured to allow flex or deformation of the catheter 500 near the connectors 504, 506. In certain implementations, the features 510, 512 may be color coded to indicate to which lumen of a multi-lumen catheter, the connectors 504, 506, correspond. In certain embodiments, the features 508, 510, 512 may take the form of approximately ⅛″ polyolefin heat shrink tubing. The position marker 514 may be a position marker as described above with reference to position marker 100.

The catheter 500 may include materials/features that allow for visualization. For example, the catheter 500 may include radiopaque features. In some of these and in other instances, the catheter 500 may be formed from or otherwise include materials that MRI-compatible.

The length L₁ of the catheter 500 may be approximately 1,300 mm with a working length L₂ of approximately 1,150 mm. The working length L₂ may be defined based on various use and design considerations. As illustrated, the working length L₂ is the distance from the distal end of the distal subassembly 560 to the distal end of the feature 508. The distance D₁ from the distal end of the feature 508 to the proximal end of the connector 506 may be approximately 150 mm. The feature 508 may have a length L₃ of approximately 35 mm and the features 510, 512 may have a length L₄ of approximately 7 mm. In certain implementations, the catheter 500 may have a length L₁ of between approximately 400 mm and approximately 1200 mm, with the working length L₂ and other measurements changed accordingly at varying scales.

FIG. 2 illustrates a sectional view taken from the region of the catheter 500 marked with cutting plane line A-A. This view illustrates a lumen 516A defined by a wall 516B. The characteristics and properties of the lumen 516A and wall 516B may be similar to the other walls and lumens described herein. As illustrated, the wall 516B has an inner diameter D₂ of approximately 0.54 mm and an outer diameter D₃ of approximately 1.14 mm.

FIG. 3 illustrates a sectional view taken from the region of the catheter 500 marked with cutting plane line B-B. This view illustrates a lumen 518A defined by an inner wall 518B and a lumen 520A defined by the space between the inner wall 518B and an outer wall 520B. The characteristics and properties of the lumens 518A, 520A and the walls 518B, 520B may be similar to the other walls and lumens described herein. The inner wall 518B may have an inner diameter D₄ of approximately 0.56 mm and an outer diameter D₅ of approximately 0.71 mm. The outer wall 520B may have an inner diameter of approximately 1.32 mm and an outer diameter of approximately 1.689 mm.

FIG. 4 illustrates an enlarged, detail view of a portion of the Y-connector 502 according to certain implementations, including tubes 522, first branch 524, and second branch 526. The tubes 522 may be hypotubes or other lengths of tubing. The tubes 522 may have a length L₅ of approximately 10 mm. In certain implementations, the first branch 524 may place the connector 504 in fluid connection with the lumen 520A and the second branch 526 may place the connector 506 in fluid connection with the lumen 518A.

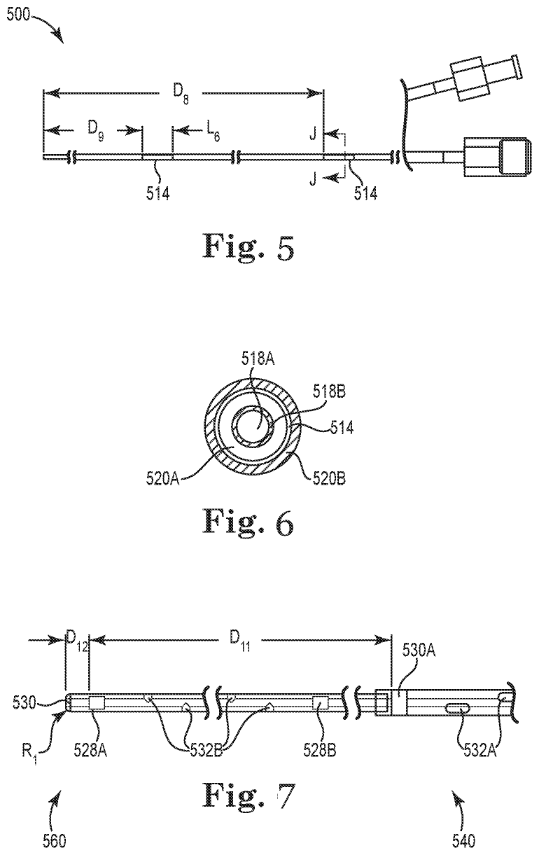

FIG. 5 illustrates the location of two position markers 514 on the catheter 500. The distal end of the first position marker 514 is located a distance D₉ of approximately 450 mm away from the distal end of the catheter 500. The distal end of the second position marker 514 is located a distance D₈ of approximately 550 mm away from the distal end of the catheter 500. The length L₄ of the position markers 514 is approximately 10 mm. In certain implementations, the bands and/or position markers (such as position markers 514) may comprise PET heat shrink tubing.

FIG. 6 illustrates a sectional view taken from the region of the catheter 500 marked with the cutting plane line J-J. This view illustrates an embodiment wherein an outer portion of the position marker 514 is substantially adjacent to an inner portion of the wall 520B. Accordingly, in this portion of this embodiment, the lumen 520A is defined by the outer portion of the wall 518B and the inner portion of the position marker 514. As illustrated, the outer wall 520B has an outer diameter D₁₀ of approximately 1.75 mm. In other instances, the position marker 514 may be disposed along the outer portion of the wall 520B, along the outer portion of the wall 518B, or along another region of the catheter 500.

FIG. 7 illustrates a portion of the catheter 500, including bands 528A, 528B, and 530A, openings 532A and 532B, and a radiused tip 530. The distal portion of the band 530A may be located a distance D₁₁ of approximately 300 mm away (e.g., or more or less, depending on the size/height of the patient) from the distal portion of the band 528A. This spacing may help to reduce local recirculation and/or help avoid sensitive nerve structures in the cervical spine. The distal end of the band 528A may be located a distance D₁₂ of approximately 2 mm away from the distal end of the radiused tip 530. The radiused tip may have a radius R₁ of approximately 0.28 mm.

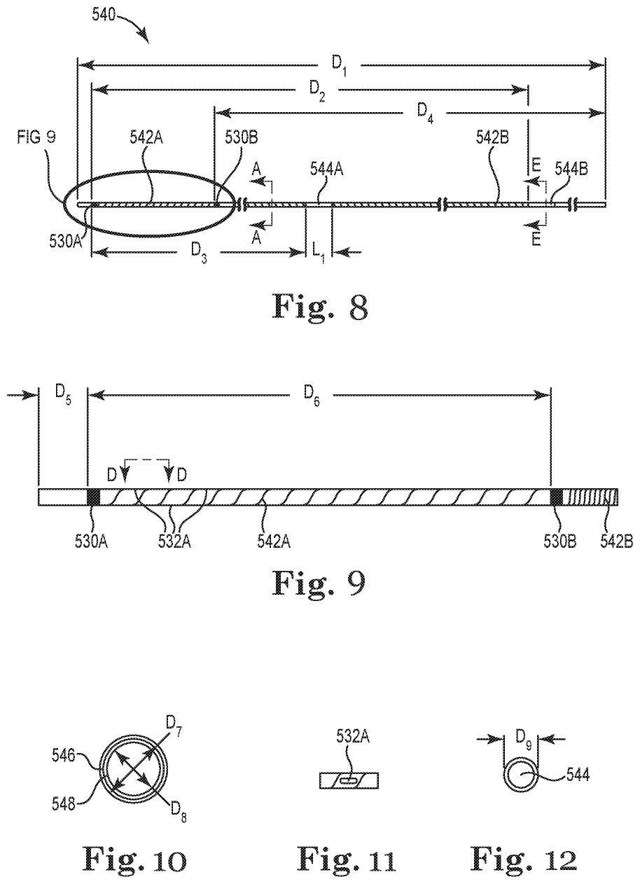

FIG. 8 illustrates a portion of the proximal subassembly 540. As illustrated, the distance D₁ from the distal end of the proximal subassembly 540 to the proximal end of the proximal subassembly 540 is approximately 893 mm. The distance D₂ from the distal end of a marker band 544B to a distal end of a band 530A is approximately 248 mm. A distance D₄ from a proximal end of the proximal subassembly 540 to the distal end of a band 530B is approximately 845 mm. A distance D₃ from a distal end of the marker band 544A to a distal end of the band 530A is approximately 148 mm. The marker bands 544A, 544B may have a length L₁ of approximately 10 mm. A portion of the proximal subassembly 540 may comprise coiled wire 542B having a coil pitch of approximately 0.018″. A portion of the proximal subassembly 540 may comprise coiled wire 542A having a coil pitch of approximately 0.095″. In certain implementations, the wires 542A, 542B may comprise approximately 0.003″ round wire spool of 304V spring temper material.

In certain implementations, the proximal subassembly 540 of the catheter 500 may have an outer diameter of between approximately 0.06″ and approximately 0.07″. This configuration may maximize the size of the catheter between layers of tissue to enable a desired level of drainage and/or suction without collapse. The thickness of the proximal subassembly 540 and other sections of the catheter 500 may be a function of a design of one or more layers of coil and sheath. The thickness may affect the stiffness and pushability of the catheter 500 and kink-resistance. In certain implementations, the diameter of an inner lumen of the catheter 500 (such as the diameter of a lumen of the proximal subassembly 540) may be chosen to provide optimum drainage and/or suction given the constraints of particular anatomy or procedures. For example, the minimum diameter of a proximal inner lumen may be chosen to be between approximately 0.025″ and approximately 0.060″.

FIG. 9 illustrates a detail view of the proximal subassembly 540 of FIG. 8. As illustrated, a portion of the proximal subassembly 540 defines a plurality of openings 532A (e.g. 2, 3, 4, 5, 6, 7, 8, 9, 10 11, 12. 13 14. 15, 16, or more openings 532A). The openings 532A may be in fluid connection with a lumen 520A of the catheter 500 and, in at least some instances, may be arranged on opposing “top” and “bottom” sides of the catheter 500. The openings 532A may be spaced with 2 coil pitch spacing of the wire 542A. The distance D₆ between the distal end of the band 530B and the distal end of the band 530A is approximately 45 mm. A distance D₅ from the distal end of the band 530A to the distal end of the proximal subassembly 540 may be approximately 3 mm. In certain implementations, the bands 530A, 530B may comprise a radiopaque band having an inner diameter of approximately 0.061″ and an outer diameter of approximately 0.064″.

FIG. 10 illustrates a sectional view taken from the region of the proximal subassembly 540 marked with the cutting plane line A-A, including a liner 546 and tubing 548. The liner 546 and the tubing 548 may be arranged such that the tubing 548 is within the liner 546. The coil 542A may be disposed between the liner 546 and the tubing 548. In certain implementations, the liner 546 may comprise approximately 0.001″ WT PTFE liner. The tubing 548 may comprise approximately 0.004″ WT polyether block amide tubing. The outer diameter D₇ of the combination tubing 548 and liner 546 may be approximately 1.69 mm. The inner diameter D₈ of the same may be approximately 1.32 mm.

FIG. 11 illustrates a detail view of a portion of the proximal subassembly 540 taken from the view of line D-D and illustrating one of the openings 532A. The illustrated opening 532A has dimensions of approximately 1.57 mm by approximately 0.56 mm. In at least some instances, the openings 532A may be oval in shape. Other shapes are contemplated. The shape of the openings 532A may be the same along the length of the proximal subassembly 540 or the shape of the openings 532A may differ along the length of the proximal subassembly 540. In at least some instances, the openings 532A may be larger than the openings 532B.

FIG. 12 illustrates a sectional view taken from the region of the proximal subassembly 540 marked with the cutting plane E-E. As illustrated the outer diameter D₉ this portion, inclusive of marker band 544 is approximately 1.75 mm.

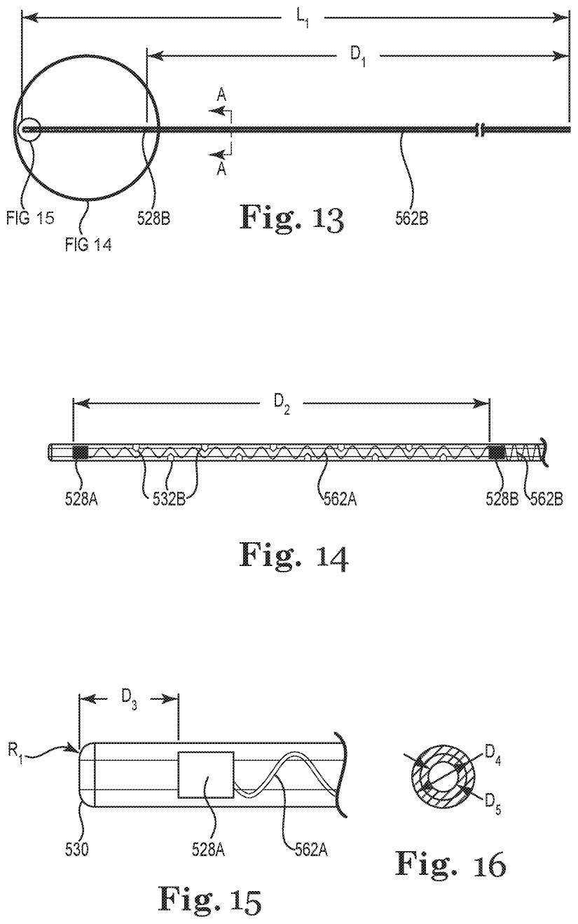

FIG. 13 illustrates a portion of the distal subassembly 560. The length L₁ of the distal subassembly 560 may be approximately 302 mm. The distance D₁ from the proximal end of the distal subassembly 560 to the distal end of a band 528B is approximately 270 mm. A portion of the distal subassembly 560 may comprise a coiled wire 562B may have a coil pitch of approximately 0.032″. This and other portions of the catheter 500 may comprise approximately 0.003″ WT nylon 12 tubing having an inner diameter of approximately 0.022″ and approximately 0.007″ WT PEBAX tubing having an inner diameter of approximately 0.04″.

FIG. 14 illustrates a detailed portion of the distal subassembly 560, including the band 528A, a plurality of openings 532B, the band 528B, a wire 562A, and the wire 562B. In certain implementations, the wires 562A, 562B may be different portions of the same wire or may be separate sections of wire. As illustrated, the wire 562A and 562B may be separated by band 528B. The wire 562A may have a coil pitch of approximately 0.065″. The wires 562A, 562B may be disposed between layers of the distal subassembly 560 and may comprise approximately 0.003″ round wire spool 304V spring temper material. The openings 532B may be spaced with 2 coil pitch spacing and arranged on a top and a bottom portion of the catheter 500 and made a fluid connection with an inner lumen 516A of the catheter 500. In at least some instances, the openings 532B may be round or substantially round. Other shapes are contemplated. The shape of the openings 532B may be the same along the length of the distal subassembly 560 or the shape of the openings 532B may differ along the length of the distal subassembly 560. A distance D₂ between the distal end of the band 528B and the distal end of the band 528A may be approximately 30 mm. The wire 562A may be disposed within this region. The bands 528A, 528B may have an inner diameter of approximately 0.032″ and an outer diameter of approximately 0.034″. The bands 528A, 528B may include a radiopaque material (e.g., the bands 528A, 528B may comprise a material such as PT/10% IR). The bands 528A, 528B may be disposed between the layers of the distal subassembly 560.

FIG. 15 illustrates a detailed portion of the distal subassembly 560, including the radiused tip 530, the band 528A, and the wire 562A. The distance from the distal end of the band 528A and the distal end of the radiused tip 530 is approximately 2 mm. The radiused tip may have a radius R₁ of approximately 0.28 mm.

FIG. 16 illustrates a sectional view taken from the region of the distal subassembly 560 marked with the cutting plane A-A. As illustrated, this section of the distal subassembly 560 has an outer diameter of approximately 1.14 mm and an inner diameter of approximately 0.53 mm.

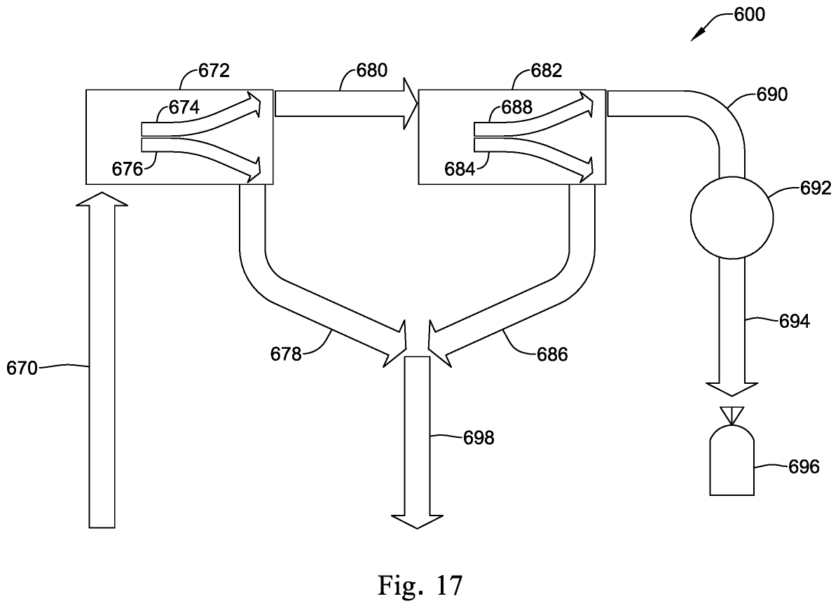

FIG. 17 schematically depicts a pump/filtration system 600 that may be utilized with the catheter 500. The catheter 500 may connect to an inlet 670 of the pump/filtration system 600. For example, the connector 504 may connect to the inlet 670 either directly or through an intermediate tube or mechanism. The inlet 670 may lead to a first filter 672. In some instances, the first filter 672 is a tangential flow filter. For example, the first filter 672 may include a 5 kDa tangential flow filter (TFF), a 100 kDa TFF, a 0.2 μm TFF, a 0.45 μm TFF, or the like. In some instances, the first filter 672 may include a dead-end filter (e.g., 5 kDa dead-end filter). In some instances the first filter 672 may include an electro-filter (e.g., a filter that excludes materials based on charge). In some instances, only one filter (e.g., the first filter 672) may be utilized. For example, the first filter 672 may be a 5 kDa filter and the first filter 672 may be the only filter. Clean CSF 676 may follow pathway 678. CSF waste 674 may follow pathway 680. The waste pathway 680 may lead to a second filter 682. In some instances, the second filter 682 is a tangential flow filter. For example, the second filter 682 may include a 5 kDa TFF, a 100 kDa TFF, a 0.2 μm TFF, a 0.45 μm TFF, or the like. In some instances, the second filter 682 may include a dead-end filter (e.g., 5 kDa dead-end filter). In some instances the second filter 682 may include an electro-filter. In at least some instances, the first filter 672 and the second filter 682 are the same size and/or type (e.g., both the first filter 672 and the second filter 682 are 100 kDa TFF). In other instances, the first filter 672 and the second filter 682 differ (e.g., the first filter 672 is a 5 kDa filter and the second filter 682 is a 100 kDa TFF filter). Clean CSF 684 may follow pathway 686. CSF waste 688 may follow pathway 690. A valve or flow metering mechanism 692 may be disposed along the waste pathway 690, before terminating at pathway 694 and a collection apparatus 696. Pathways 678 and 686 may merge into a return outlet 698, which may connect to the connector 506 of the catheter 500 (e.g., either directly or through an intermediate tube).

In use, the catheter 500 may be disposed within the cerebrospinal space (e.g., such as along lumbar cerebrospinal space). CSF may be removed/aspirated using the catheter 500 (e.g., via the lumen 520A) and the pump/filtration system 600. The aspirated fluid may be filtered using the pump/filtration system 600 and the filtered/conditioned CSF may be returned to the patient using the catheter 500 (e.g., via the lumen 518A) and the pump/filtration system 600. In some instances, a second catheter 500 (that may be similar in form and function to the catheter 500) may be disposed in a portion of the cranial CNS such as within a ventricle. The second catheter 500 may be used to remove/aspirate cerebrospinal fluid from a cranial region (e.g., a ventricle), condition/filter the cerebrospinal fluid using the pump/filtration system 600, and return the conditioned/filtered cerebrospinal fluid to a region at or adjacent to the cranial region. In some of these and in other instance, the second catheter 500 may be used to infuse a drug (e.g., a chemotherapy drug such as methotrexate) into the cranial region. The catheter 500 (e.g., in the cerebrospinal space) and the second catheter 500 (in the ventricle) may be used together or they may be used alternately. Using a catheter 500 in both the cerebrospinal space and in the ventricle, both for aspiration and infusion, may form a cranial-lumbar loop that may improve circulation of cerebrospinal fluid throughout the CNS.

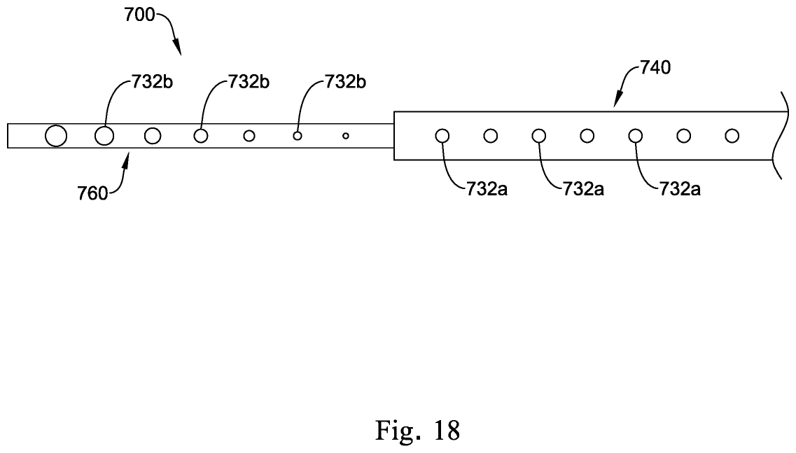

For a number of reasons, when aspirating fluid and/or infusing fluid along the CNS it may be desirable to aspirate/infuse fluid uniformly (e.g., approximately uniformly) along the catheter and/or along the aspiration/infusion openings. FIGS. 18-22 depict example catheters, similar in form and function to other catheters disclosed herein, that are designed to infuse and/or aspirate fluid uniformly. Some details regarding these catheters are disclosed herein.

FIG. 18 depicts another example catheter 700 that may be similar in form and function to other catheters disclosed herein. The catheter 700 may include a proximal subassembly 740. In general, the proximal subassembly 740 may include at least some of the structures and features similar to those of the proximal subassembly 540. For example, the proximal subassembly 740 may include or otherwise take the form of a tube. The catheter 700 may also include a distal subassembly 760. In general, the distal subassembly 760 may include at least some of the structures and features similar to those of the distal subassembly 560. For example, the distal subassembly 760 may include or otherwise take the form of a tube. An infusion lumen (not shown in FIG. 18, but is generally similar to the lumen 518A) may be disposed along or otherwise formed in the distal subassembly 760. An aspiration lumen (not shown in FIG. 18, but is generally similar to the lumen 520A) may be disposed defined between the outer surface of the distal subassembly 760 (e.g. the outer surface of the tubular member that is part of or otherwise forms the distal subassembly 760) and the inner surface of the proximal subassembly 740 (e.g. the in surface of the tubular member that is part of or otherwise forms the proximal subassembly 740).

The proximal subassembly 740 may include a plurality of openings or apertures 732A formed therein. In general, the openings 732A are designed so that fluid in the CNS (e.g., CSF fluid) can be removed/aspirated from the CNS, for example when the catheter 700 is coupled to pump/filtration system 600. The distal subassembly 760 may also include a plurality of openings or apertures 732B. In general, the openings 732A are designed so that fluid (e.g., CSF fluid that is filtered, conditioned, treated, and/or the like by the pump/filtration system 600) can be returned/infused into the CNS, for example when the catheter 700 is coupled to pump/filtration system 600.

As indicated above, the catheter 700 may be designed to infuse and/or aspirate fluid uniformly. In some instances, the catheter 700 may utilize the openings 732B to infuse fluid (e.g., CSF fluid that is filtered, conditioned, treated, and/or the like by the pump/filtration system 600) into the CNS. For the purposes of this disclosure, infusing fluid uniformly may be understood to mean that when fluid is infused through the openings 732B, relatively equal volumes of fluid pass through each of the openings 732B. In other words, the majority of the volume of the infused fluid does not tend to pass through the more proximal of the openings 732B but rather the volume of the infused fluid is relatively evenly or uniformly distributed among the openings 732B (e.g., all the openings 732B).

In some instances, the openings 732B the openings 732B may get larger as the openings 732B move more distally. This transition may be continuous (e.g., each subsequent opening 732B may get larger), stepped (e.g., groups of openings 732B are the same size with more distal groupings of openings 732B being larger), regular (e.g., the transition in size proceeds in a predictable pattern), irregular (e.g., the transition in size proceeds in a random fashion), or the like.

The change in size may be the result of the openings 732B being the same shape but of different (e.g., increasing distally) size. For example, in some instances, the first or most proximal opening(s) 732B may be generally round in shape and subsequent openings are also round but are larger (e.g., surface area that the opening spans). In some instances, subsequent distal openings 732B may increase in size by 1-100%, or about 5-50%, or about 10-25%. Openings, and the distances between openings, can be tailored to produce non-uniform hydrodynamic resistance along the tube to either deliver or remove equal fluid flow rate from each hole. Hydrodynamic resistance will be based on the pressure head loss within the tubing and losses due to the specific hole geometries. These losses can be tuned to result in desired flow rate(s) from each hole.

In some of these and in other instances, the change in size may be the result of the openings 732B changing shape in the distal direction. For example, the first or most proximal opening 732B may be generally round in shape and subsequent openings may increase by the shape of the openings 732B changing to a different shape that is larger or otherwise has a larger surface area than more proximal openings. For example, the first opening(s) 732B may be round and subsequent opening 732B may transition to larger, more oval shape. These holes may be machined to create smooth surface transition (rounded) that reduces cell adherence to the surface. They may also be positioned in a non-uniform fashion along the catheter length.

In some of these and in other instances, the openings 732B may be of the same of similar size/shape but the number of openings 732B per unit length may increase in the distal direction. In other words, the density of the openings 732B may increase and they may also be located at different angular positions around the catheter lumen to reduce propensity for clogging and or improve removal or delivery of solutes to the CSF.

The openings 732B may be distributed along the distal subassembly 760 in a number of manners. For example, the distal subassembly 760 may include one or more rows of axially-aligned rows of openings 732B. In some of these and in other instances, at least some of the openings 732B may be arranged circumferentially about the distal subassembly 760. In one example, at least some of the openings 732B are arranged in a helical manner about the distal subassembly 760.

In use, the catheter 700 may be disposed within the cerebrospinal space (e.g., such as along lumbar cerebrospinal space). CSF may be removed/aspirated using the openings 732A of the proximal subassembly 740 and sent to the pump/filtration system 600. The aspirated fluid may be filtered using the pump/filtration system 600 (e.g., to remove blood, foreign agents, chemicals/drugs, and/or the like) and the filtered/conditioned CSF may be returned to the patient (e.g., along a lumbar or other region) using the openings 732B of the distal subassembly 760 (e.g., and the pump/filtration system 600). In some instances, a drug or therapeutic may also be infused. The openings 732B may substantially uniformly distribute filtered/conditioned CSF to the CNS along the length of the distal subassembly 760. The use of other catheters disclosed herein may be analogous. In some other instances, the catheter 700 may be added and/or coupled to an existing (e.g., implanted) CSF shut such as a lumboperitoneal shut.

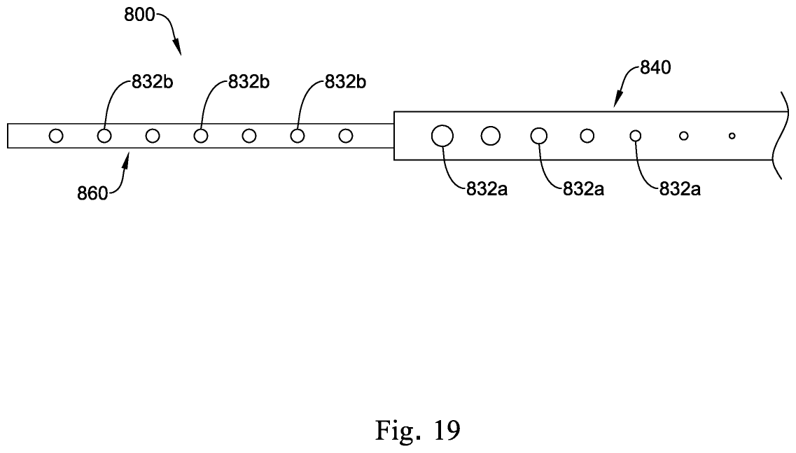

FIG. 19 depicts another example catheter 800 that may be similar in form and function to other catheters disclosed herein. The catheter 800 may include a proximal subassembly 840. In general, the proximal subassembly 840 may include at least some of the structures and features similar to those of the proximal subassembly 540. For example, the proximal subassembly 840 may include or otherwise take the form of a tube. The catheter 800 may also include a distal subassembly 860. In general, the distal subassembly 860 may include at least some of the structures and features similar to those of the distal subassembly 560. For example, the distal subassembly 860 may include or otherwise take the form of a tube. An infusion lumen (not shown in FIG. 19, but is generally similar to the lumen 518A) may be disposed along or otherwise formed in the distal subassembly 860. An aspiration lumen (not shown in FIG. 19, but is generally similar to the lumen 520A) may be disposed defined between the outer surface of the distal subassembly 860 (e.g. the outer surface of the tubular member that is part of or otherwise forms the distal subassembly 860) and the inner surface of the proximal subassembly 840 (e.g. the in surface of the tubular member that is part of or otherwise forms the proximal subassembly 840).

The proximal subassembly 840 may include a plurality of openings or apertures 832A formed therein. In general, the openings 832A are designed so that fluid in the CNS (e.g., CSF fluid) can be removed/aspirated from the CNS, for example when the catheter 800 is coupled to pump/filtration system 600. The distal subassembly 860 may also include a plurality of openings or apertures 832B. In general, the openings 832A are designed so that fluid (e.g., CSF fluid that is filtered, conditioned, treated, and/or the like by the pump/filtration system 600) can be returned/infused into the CNS, for example when the catheter 800 is coupled to pump/filtration system 600.

As indicated above, the catheter 800 may be designed to infuse and/or aspirate fluid uniformly. In some instances, the catheter 800 may utilize the openings 832A to aspirate fluid uniformly from the CNS. For the purposes of this disclosure, aspirating fluid uniformly may be understood to mean that when fluid is aspirated through the openings 832A, relatively equal volumes of fluid pass through each of the openings 832A.

In some instances, the openings 832A the openings 832A may get larger as the openings 832A move more distally. This transition may be continuous (e.g., each subsequent opening 832A may get larger), stepped (e.g., groups of openings 832A are the same size with more distal groupings of openings 832A being larger), regular (e.g., the transition in size proceeds in a predictable pattern), irregular (e.g., the transition in size proceeds in a random fashion), or the like.

The change in size may be the result of the openings 832A being the same shape but of different (e.g., increasing distally) size. For example, in some instances, the first or most proximal opening(s) 832A may be generally round in shape and subsequent openings are also round but are larger (e.g., surface area that the opening spans). In some instances, subsequent distal openings 832A may increase in size by 1-100%, or about 5-50%, or about 10-25%.

In some of these and in other instances, the change in size may be the result of the openings 832A changing shape in the distal direction. For example, the first or most proximal opening 832A may be generally round in shape and subsequent openings may increase by the shape of the openings 832A changing to a different shape that is larger or otherwise has a larger surface area than more proximal openings. For example, the first opening(s) 832A may be round and subsequent opening 832A may transition to larger, more oval shape.

In some of these and in other instances, the openings 832A may be of the same or similar size/shape but the number of openings 832A per unit length may increase in the distal direction. In other words, the density of the openings 832A may increase.

The openings 832A may be distributed along the proximal subassembly 840 in a number of manners. For example, the proximal subassembly 840 may include one or more rows of axially-aligned rows of openings 832A. In some of these and in other instances, at least some of the openings 832A may be arranged circumferentially about the proximal subassembly 840. In one example, at least some of the openings 832A are arranged in a helical manner about the proximal subassembly 840.

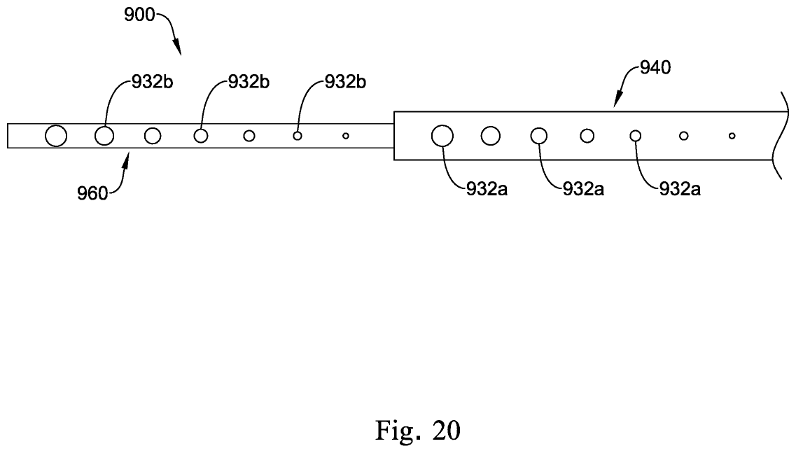

FIG. 20 depicts another example catheter 900 that may be similar in form and function to other catheters disclosed herein. The catheter 900 may include a proximal subassembly 940. In general, the proximal subassembly 940 may include at least some of the structures and features similar to those of the proximal subassembly 540. For example, the proximal subassembly 940 may include or otherwise take the form of a tube. The catheter 900 may also include a distal subassembly 960. In general, the distal subassembly 960 may include at least some of the structures and features similar to those of the distal subassembly 560. For example, the distal subassembly 960 may include or otherwise take the form of a tube. An infusion lumen (not shown in FIG. 20, but is generally similar to the lumen 518A) may be disposed along or otherwise formed in the distal subassembly 960. An aspiration lumen (not shown in FIG. 20, but is generally similar to the lumen 520A) may be disposed defined between the outer surface of the distal subassembly 960 (e.g. the outer surface of the tubular member that is part of or otherwise forms the distal subassembly 960) and the inner surface of the proximal subassembly 940 (e.g. the in surface of the tubular member that is part of or otherwise forms the proximal subassembly 940).

The proximal subassembly 940 may include a plurality of openings or apertures 932A formed therein. In general, the openings 932A are designed so that fluid in the CNS (e.g., CSF fluid) can be removed/aspirated from the CNS, for example when the catheter 900 is coupled to pump/filtration system 600. The distal subassembly 960 may also include a plurality of openings or apertures 932B. In general, the openings 932A are designed so that fluid (e.g., CSF fluid that is filtered, conditioned, treated, and/or the like by the pump/filtration system 600) can be returned/infused into the CNS, for example when the catheter 900 is coupled to pump/filtration system 600.

As indicated above, the catheter 900 may be designed to infuse and/or aspirate fluid uniformly. In some instances, the catheter 900 may utilize the openings 932A/932B to aspirate/infuse fluid uniformly from the CNS. For the purposes of this disclosure, aspirating/infusing fluid uniformly may be understood to mean that when fluid is aspirated/infused through the openings 932A/932B, relatively equal volumes of fluid pass through each of the openings 932A/932B. In this example, the openings 932B in the distal subassembly 960 get larger (e.g., in the manner described with respect to the distal subassembly 760 and/or openings 732B) and the openings 932A in the proximal subassembly 940 get larger (e.g., in the manner described with respect to the proximal subassembly 840 and/or openings 832A)

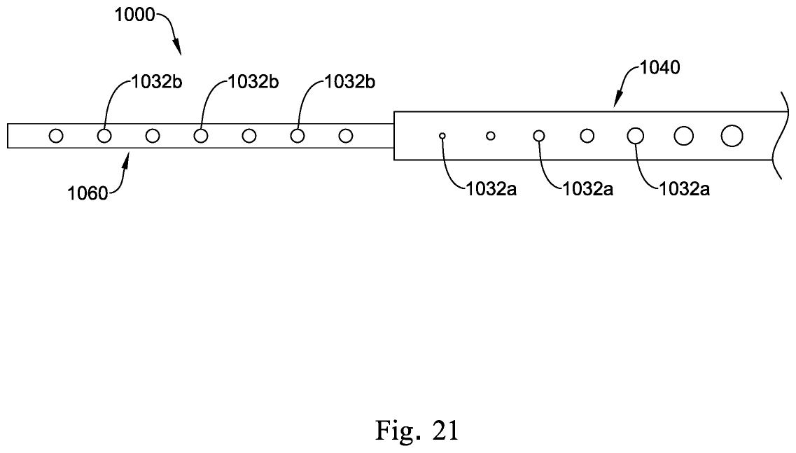

FIG. 21 depicts another example catheter 1000 that may be similar in form and function to other catheters disclosed herein. The catheter 1000 may include a proximal subassembly 1040. In general, the proximal subassembly 1040 may include at least some of the structures and features similar to those of the proximal subassembly 540. For example, the proximal subassembly 1040 may include or otherwise take the form of a tube. The catheter 1000 may also include a distal subassembly 1060. In general, the distal subassembly 1060 may include at least some of the structures and features similar to those of the distal subassembly 560. For example, the distal subassembly 1060 may include or otherwise take the form of a tube. An infusion lumen (not shown in FIG. 21, but is generally similar to the lumen 518A) may be disposed along or otherwise formed in the distal subassembly 1060. An aspiration lumen (not shown in FIG. 21, but is generally similar to the lumen 520A) may be disposed defined between the outer surface of the distal subassembly 1060 (e.g. the outer surface of the tubular member that is part of or otherwise forms the distal subassembly 1060) and the inner surface of the proximal subassembly 1040 (e.g. the in surface of the tubular member that is part of or otherwise forms the proximal subassembly 1040).

The proximal subassembly 1040 may include a plurality of openings or apertures 1032A formed therein. In general, the openings 1032A are designed so that fluid in the CNS (e.g., CSF fluid) can be removed/aspirated from the CNS, for example when the catheter 1000 is coupled to pump/filtration system 600. The distal subassembly 1060 may also include a plurality of openings or apertures 1032B. In general, the openings 1032A are designed so that fluid (e.g., CSF fluid that is filtered, conditioned, treated, and/or the like by the pump/filtration system 600) can be returned/infused into the CNS, for example when the catheter 1000 is coupled to pump/filtration system 600.

As indicated above, the catheter 1000 may be designed to infuse and/or aspirate fluid uniformly. In some instances, the catheter 1000 may utilize the openings 1032A to aspirate fluid uniformly from the CNS. For the purposes of this disclosure, aspirating fluid uniformly may be understood to mean that when fluid is aspirated through the openings 1032A, relatively equal volumes of fluid pass through each of the openings 1032A.

In some instances, the openings 1032A the openings 1032A may get larger as the openings 1032A move more proximally. This transition may be continuous (e.g., each subsequent opening 1032A may get larger), stepped (e.g., groups of openings 1032A are the same size with more proximal groupings of openings 1032A being larger), regular (e.g., the transition in size proceeds in a predictable pattern), irregular (e.g., the transition in size proceeds in a random fashion), or the like.

The change in size may be the result of the openings 1032A being the same shape but of different (e.g., increasing proximally) size. For example, in some instances, the first or most proximal opening(s) 1032A may be generally round in shape and subsequent openings are also round but are larger (e.g., surface area that the opening spans). In some instances, subsequent proximal openings 1032A may increase in size by 1-100%, or about 5-50%, or about 10-25%.

In some of these and in other instances, the change in size may be the result of the openings 1032A changing shape in the proximal direction. For example, the first or most distal opening 1032A may be generally round in shape and subsequent openings may increase by the shape of the openings 1032A changing to a different shape that is larger or otherwise has a larger surface area than more distal openings. For example, the first opening(s) 1032A may be round and subsequent opening 1032A may transition to larger, more oval shape.

In some of these and in other instances, the openings 1032A may be of the same of similar size/shape but the number of openings 1032A per unit length may increase in the proximal direction. In other words, the density of the openings 1032A may increase.

The openings 1032A may be distributed along the proximal subassembly 1040 in a number of manners. For example, the proximal subassembly 1040 may include one or more rows of axially-aligned rows of openings 1032A. In some of these and in other instances, at least some of the openings 1032A may be arranged circumferentially about the proximal subassembly 1040. In one example, at least some of the openings 1032A are arranged in a helical manner about the proximal subassembly 1040.

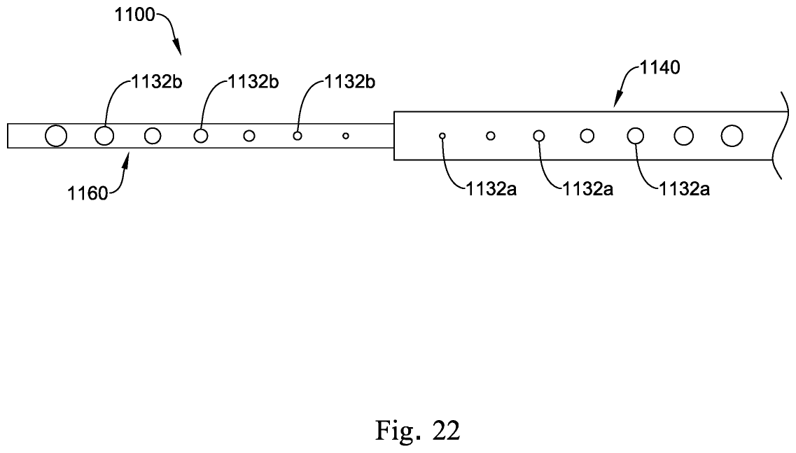

FIG. 22 depicts another example catheter 1100 that may be similar in form and function to other catheters disclosed herein. The catheter 1100 may include a proximal subassembly 1140. In general, the proximal subassembly 1140 may include at least some of the structures and features similar to those of the proximal subassembly 540. For example, the proximal subassembly 1140 may include or otherwise take the form of a tube. The catheter 1100 may also include a distal subassembly 1160. In general, the distal subassembly 1160 may include at least some of the structures and features similar to those of the distal subassembly 560. For example, the distal subassembly 1160 may include or otherwise take the form of a tube. An infusion lumen (not shown in FIG. 22, but is generally similar to the lumen 518A) may be disposed along or otherwise formed in the distal subassembly 1160. An aspiration lumen (not shown in FIG. 21, but is generally similar to the lumen 520A) may be disposed defined between the outer surface of the distal subassembly 1160 (e.g. the outer surface of the tubular member that is part of or otherwise forms the distal subassembly 1160) and the inner surface of the proximal subassembly 1140 (e.g. the in surface of the tubular member that is part of or otherwise forms the proximal subassembly 1140).

The proximal subassembly 1140 may include a plurality of openings or apertures 1132A formed therein. In general, the openings 1132A are designed so that fluid in the CNS (e.g., CSF fluid) can be removed/aspirated from the CNS, for example when the catheter 1100 is coupled to pump/filtration system 600. The distal subassembly 1160 may also include a plurality of openings or apertures 1132B. In general, the openings 1132A are designed so that fluid (e.g., CSF fluid that is filtered, conditioned, treated, and/or the like by the pump/filtration system 600) can be returned/infused into the CNS, for example when the catheter 1100 is coupled to pump/filtration system 600.

As indicated above, the catheter 1100 may be designed to infuse and/or aspirate fluid uniformly. In some instances, the catheter 1100 may utilize the openings 1132A/1132B to aspirate/infuse fluid uniformly from the CNS. For the purposes of this disclosure, aspirating/infusing fluid uniformly may be understood to mean that when fluid is aspirated/infused through the openings 1132A/1132B, relatively equal volumes of fluid pass through each of the openings 1132A/1132B. In this example, the openings 1132B in the distal subassembly 1160 get larger (e.g., in the manner described with respect to the distal subassembly 760 and/or openings 732B) and the openings 1132A in the proximal subassembly 1140 get larger (e.g., in the manner described with respect to the proximal subassembly 840 and/or openings 832A).

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system for processing cerebrospinal fluid, the system comprising:
a catheter having a proximal subassembly and a distal subassembly;
a pump and filtration system coupled to the catheter;
wherein an infusion lumen for infusing cerebrospinal fluid filtered by the pump and filtration system is defined along the distal subassembly;
wherein the distal subassembly defines a plurality of infusion openings in fluid communication with the infusion lumen; and
wherein the infusion openings increase in size distally along the distal subassembly.

2. The system of claim 1, wherein at least some of the infusion openings have a round shape.

3. The system of claim 1, wherein at least some of the infusion openings have a non-circular shape.

4. The system of claim 1, wherein all of the infusion openings have the same shape.

5. The system of claim 1, where at least some of the infusion openings differ in shape.

6. The system of claim 1, wherein the plurality of infusion openings include a row of axially-aligned infusion openings.

7. The system of claim 1, wherein at least some of the plurality of infusion openings extend circumferentially about the distal subassembly.

8. The system of claim 1, wherein the proximal subassembly includes a plurality of aspiration openings.

9. The system of claim 8, wherein at least some of the aspiration openings have a round shape.

10. The system of claim 8, wherein at least some of the aspiration openings have a non-circular shape.

11. The system of claim 8, wherein all of the aspiration openings have the same shape.

12. The system of claim 8, where at least some of the aspiration openings differ in shape.

13. The system of claim 8, wherein the plurality of aspiration openings include a row of axially-aligned aspiration openings.

14. The system of claim 8, wherein at least some of the plurality of aspiration openings extend circumferentially about the proximal subassembly.

15. The system of claim 8, wherein the aspiration openings increase in size distally along the proximal subassembly.

16. The system of claim 8, wherein the aspiration openings increase in size proximally along the proximal subassembly.

17. A system for processing cerebrospinal fluid, the system comprising:
a catheter having a proximal subassembly and a distal subassembly;
a pump and filtration system coupled to the catheter;
wherein an infusion lumen for infusing cerebrospinal fluid filtered by the pump and filtration system is defined along the distal subassembly;
wherein the distal subassembly defines a plurality of infusion openings in fluid communication with the infusion lumen;
wherein the infusion openings increase in size distally along the distal subassembly;
wherein an aspiration lumen for aspirating cerebrospinal fluid is defined along the distal subassembly; and
wherein the proximal subassembly defines a plurality of aspiration openings in fluid communication with the aspiration lumen.

18. The system of claim 17, wherein the aspiration openings increase in size distally along the proximal subassembly.

19. The system of claim 17, wherein the aspiration openings increase in size proximally along the proximal subassembly.

20. A system for processing cerebrospinal fluid, the system comprising:
a catheter having a proximal subassembly and a distal subassembly;
a pump and filtration system coupled to the catheter;
wherein an infusion lumen for infusing cerebrospinal fluid filtered by the pump and filtration system is defined along the distal subassembly;
wherein the distal subassembly defines a plurality of infusion openings in fluid communication with the infusion lumen;
wherein the infusion openings increase in size distally along the distal subassembly;
wherein an aspiration lumen for aspirating cerebrospinal fluid is defined along the distal subassembly;
wherein the proximal subassembly defines a plurality of aspiration openings in fluid communication with the aspiration lumen;
wherein the aspiration openings change in size along the proximal subassembly.
